(19) United States
(12) Patent Application Publication
(10) Pub. No.: US 2024/0173511 A1
(43) Pub. Date: May 30, 2024

(54) CPAP APPARATUS

(71) Applicant: Murata Manufacturing Co., Ltd., Kyoto (JP)

(72) Inventor: Atsushi HATTORI, Kyoto (JP)

(21) Appl. No.: 18/496,344

(22) Filed: Oct. 27, 2023

(30) Foreign Application Priority Data
Nov. 30, 2022 (JP) 2022-191514

(51) Int. Cl. A61M 16/10; A61M 16/00

(52) U.S. Cl. CPC A61M 16/1075; A61M 16/0003; A61M 16/021

(57) ABSTRACT

A box body includes a lower wall, and a side wall and a side wall that are arranged with a space therebetween in which a humidification chamber is capable of being disposed. A cover includes an upper wall having a plate shape and a side wall connected to the upper wall and rotatably connected to the side wall. The humidification chamber includes a main body and a tubular body. The main body stores liquid and has a box shape that is open upward, and the main body includes a chamber side wall and a chamber side wall. The tubular body is mounted on the main body, passes through the chamber side wall, and partially protrudes outside. The humidification chamber is accommodated in the box body with the side wall and the chamber side wall facing one another and with the side wall and the chamber side wall facing one another.

### CROSS REFERENCE TO RELATED APPLICATION

This application claims priority from Japanese Patent Application No. 2022-191514 filed on Nov. 30, 2022. The content of this application is incorporated herein by reference in its entirety.

### BACKGROUND OF THE DISCLOSURE

### 1. Field of the Disclosure

The present disclosure relates to a CPAP apparatus including a water storage tank.

### 2. Description of the Related Art

A humidified pressure support therapy device is described in the specification of Japanese Patent No. 6814137. The device of the specification of Japanese Patent No. 6814137 includes a main body and a humidification chamber accommodatable in the main body. The main body includes a cover and a decoupler mechanism.

The decoupler mechanism includes a link mechanism constituted by a lifting tray and a track. When the cover is opened and closed, the link mechanism operates and moves the humidification chamber into the main body. Thus, the humidification chamber is disposed at appropriate positions when the cover is closed (when in use) and when the cover is opened (when water replenishment is performed).

### BRIEF SUMMARY OF THE DISCLOSURE

However, the device in the specification of Japanese Patent No. 6814137 is required to include such a link mechanism, thereby having an intricate device structure and a large size.

Thus, a possible benefit of the present disclosure is to achieve downsizing with a simple configuration while a humidification chamber is disposed at appropriate positions when in use and when liquid replenishment is performed.

A CPAP apparatus of the disclosure includes; a main body including a box body whose upper surface side is open, a cover that covers the upper surface side of the box body, and a first opening and a second opening; and a humidification chamber accommodatable inside the main body and disposed between the first opening and the second opening.

The box body includes a lower wall, and a first side wall and a second side wall that are arranged with a space therebetween in which the humidification chamber is capable of being disposed. The cover includes a main wall having a plate shape and a cover-side side wall connected to the main wall and rotatably connected to the first side wall. The humidification chamber includes a main body and a tubular body. The main body stores liquid and has a box shape that is open upward, and the main body includes a third side wall and a fourth side wall. The tubular body passes through the fourth side wall.

The humidification chamber is accommodated in the box body with the first side wall and the third side wall facing one another and with the second side wall and the fourth side wall facing one another. The second side wall of the box body has a through hole through which the tubular body and the inside of the main body are communicable with one another with the humidification chamber being accommodated in the box body. The cover-side side wall includes a pressing portion that is in contact with the third side wall when rotated.

In this configuration, when, with the humidification chamber being mounted on the box body, the cover is rotated in a direction in which the cover is closed, the pressing portion of the cover-side side wall pushes the humidification chamber to the second side wall side. Thus, a distal end portion of the tubular body of the humidification chamber abuts on the second side wall, and the opening of the tubular body and the through hole of the second side wall or a wall of a cylindrical body that is inserted in the through hole are communicated while being sealed from outside. The humidification chamber is fixed to the main body in this state.

According to the disclosure, downsizing is achieved with a simple configuration while the humidification chamber is disposed at appropriate positions when in use and when water replenishment is performed.

### BRIEF DESCRIPTION OF THE SEVERAL VIEWS OF THE DRAWINGS

FIG. 1 is an external perspective view of a CPAP apparatus according to an embodiment of the present disclosure;

FIG. 2 is an external perspective view of the CPAP apparatus according to the embodiment of the present disclosure;

FIG. 3 is an external perspective view of a main unit according to the embodiment of the present disclosure;

FIG. 4 is an external perspective view of a base unit according to the embodiment of the present disclosure;

FIG. 5 is a top view of the base unit according to the embodiment of the present disclosure;

FIG. 6 is a side view of the base unit according to the embodiment of the present disclosure;

FIG. 7 is a sectional side view schematically illustrating the configuration of the base unit according to the embodiment of the present disclosure;

FIG. 8 is a sectional side view schematically illustrating the configuration of the base unit according to the embodiment of the present disclosure;

FIGS. 9A, 9B, and 9C are each a partial sectional side view of an example of the movement of a cover relative to a box body;

FIG. 10 is a four orthogonal view of a humidification chamber 40 according to the embodiment of the present disclosure;

FIG. 11 is a partial sectional side view of a first state of the mounting state of the humidification chamber on the base unit;

FIG. 12A is a partial sectional side view of a second state of the mounting state of the humidification chamber on the base unit, FIG. 12B is a sectional side view of an enlarged connection area of a side wall of the cover and the humidification chamber in the second state, and FIG. 12C is a top view of the enlarged connection area of the side wall of the cover and the humidification chamber in the second state; and

FIG. 13A is a partial sectional side view of a third state of the mounting state of the humidification chamber on the base unit, FIG. 13B is a sectional side view of the enlarged connection area of the side wall of the cover and the humidification chamber in the third state, and FIG. 13C is a top view of the enlarged connection area of the side wall of the cover and the humidification chamber in the third state.

### DETAILED DESCRIPTION OF THE DISCLOSURE

A CPAP apparatus according to an embodiment of the present disclosure will be described with reference to the drawings.

### Overview of CPAP Apparatus 10

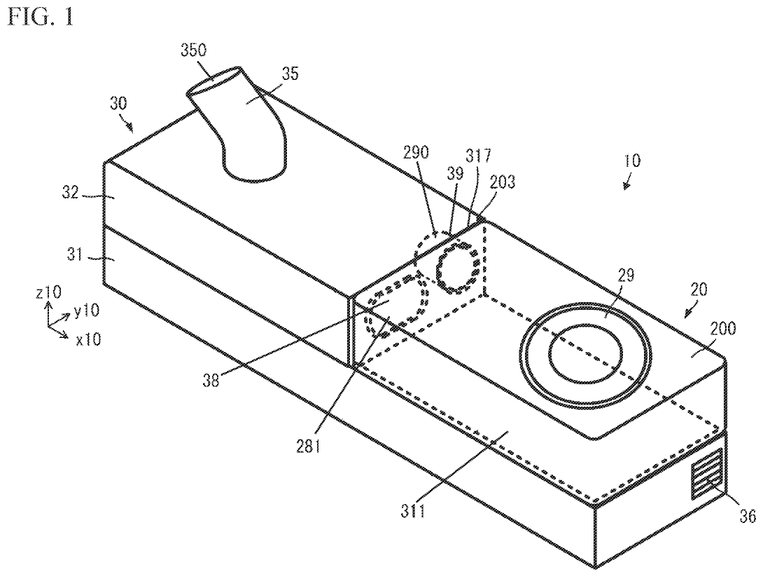

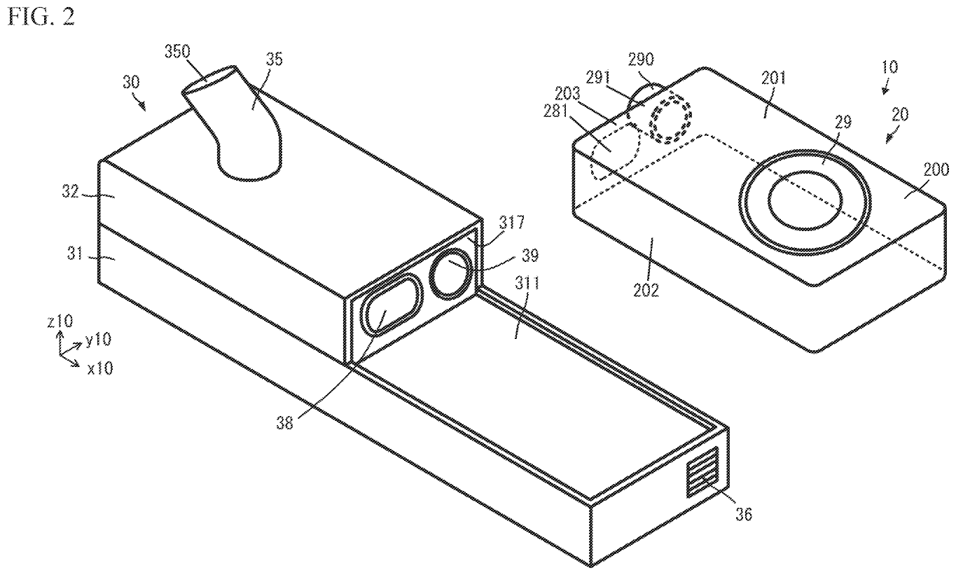

FIGS. 1 and 2 are each an external perspective view of the CPAP apparatus according to the embodiment of the present disclosure. FIG. 1 illustrates a state in which a main unit is mounted on a base unit, and FIG. 2 illustrates a state in which the main unit is removed from the base unit.

As FIGS. 1 and 2 illustrate, such a CPAP apparatus 10 includes a main unit 20 and a base unit 30.

The outside shape of the main unit 20 is a cuboid and has a height shorter than the length in the longitudinal direction and the length in the lateral direction. Note that such a cuboid in the present embodiment is not limited to a perfect cuboid and includes one whose apex or edge is chamfered.

The base unit 30 is, in plan view, a rectangular having a longitudinal direction and a lateral direction. The base unit 30 includes a tall part and a short part, and the tall part is taller than the short part. That is, the base unit 30 has an outside shape in which the tall part having a cuboid shape and the short part having a cuboid shape are connected in the longitudinal direction of the base unit 30.

The main unit 20 can be mounted on an upper surface of the short part of the base unit 30. With the main unit 20 being mounted on the base unit 30, the outside shape of the CPAP apparatus 10 is a cuboid.

In a state in which the main unit 20 is mounted on the base unit 30 (a mounted state), a gas flow passage of the main unit 20 and a gas flow passage of the base unit 30 are communicated. In addition, a first electric circuit of the main unit 20 and a second electric circuit of the base unit 30 are electrically connected. Thus, the main unit 20 and the base unit 30 achieve a function of the CPAP apparatus 10 in cooperation with one another. Note that, with the main unit 20 being removed from the base unit 30, the main unit 20 operates individually to achieve a function of the CPAP apparatus 10.

### Main Unit 20

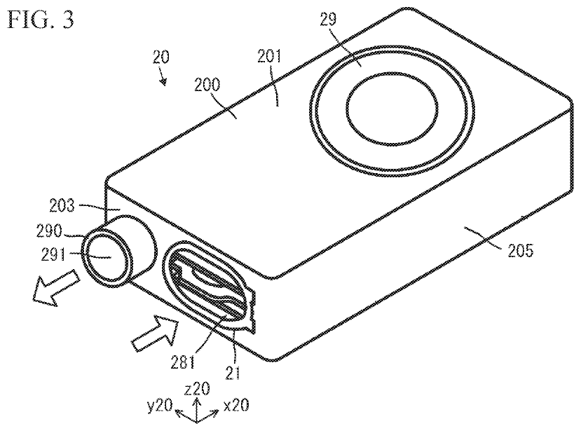

FIG. 3 is an external perspective view of the main unit according to the embodiment of the present disclosure. As FIG. 3 illustrates, the main unit 20 includes a housing 200.

The outside shape of the housing 200 is a cuboid having a longitudinal direction (the x20-axis direction) and a lateral direction (the y20-axis direction) and having a predetermined height (the length in the z20-axis direction). The housing 200 includes an upper wall 201, a lower wall 202, a side wall 203, a side wall 204, a side wall 205, and a side wall 206.

The upper wall 201 and the lower wall 202 have substantially the same outside shape and face one another. The side wall 203 and the side wall 204 have substantially the same outside shape and face one another. The side wall 203 and the side wall 204 are parallel to the lateral direction of the main unit 20. The side wall 205 and the side wall 206 have substantially the same outside shape and face one another. The side wall 205 and the side wall 206 are parallel to the longitudinal direction of the main unit 20.

The housing 200 has an inside space S200 surrounded by the upper wall 201, the lower wall 202, the side wall 203, the side wall 204, the side wall 205, and the side wall 206. In the inside space S200, the main unit-side electric circuit including a fan (for example, a motor) is disposed (not illustrated).

The side wall 203 has an inlet port 281 and an exhaust port 291. The inlet port 281 and the exhaust port 291 pass through the side wall 203 in the thickness direction. The inlet port 281 and the exhaust port 291 are arranged adjacent to one another in the width direction of the side wall 203 (the y20-axis direction). The inside space S200 of the housing 200 is communicated with an outside space of the housing 200 through the inlet port 281 and the exhaust port 291.

A cylindrical body 290 is mounted on the side wall 203. The cylindrical body 290 has a shape protruding from the outer surface of the side wall 203. A cavity at the center of the cylindrical body 290 is connected to the exhaust port 291 formed in the side wall 203, and the cavity of the cylindrical body 290 also functions as the exhaust port 291.

An air filter is disposed at the inlet port 281. A filter cover 21 is mounted on the outer surface side of the air filter. The air filter is sandwiched between the filter cover 21 and the side wall 203 to be fixed over the inlet port 281.

An operation button 29 is disposed on the upper wall 201. The operation button 29 is connected to the main unit-side electric circuit. Operations such as starting and stopping of the main unit-side electric circuit are controlled by operating the operation button 29.

### Base Unit 30

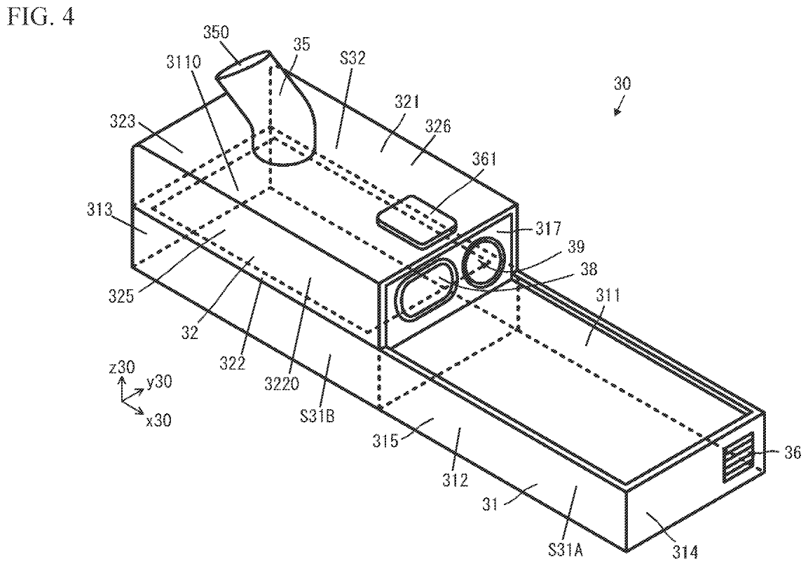

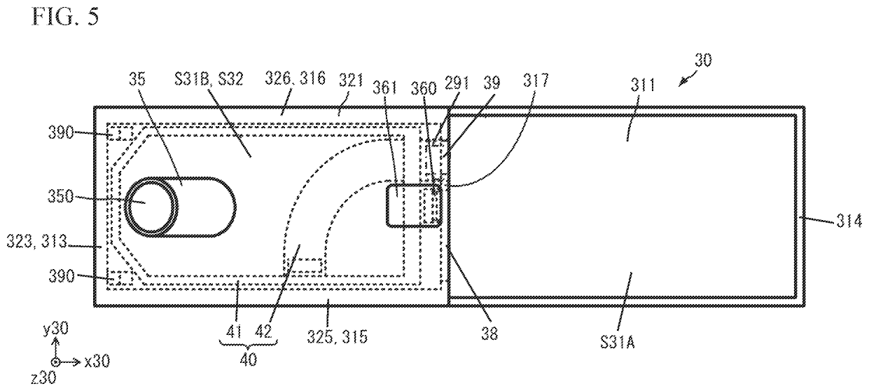

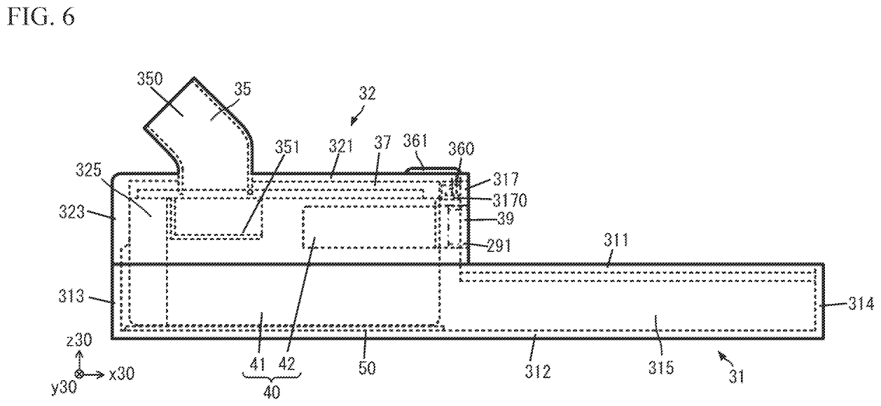

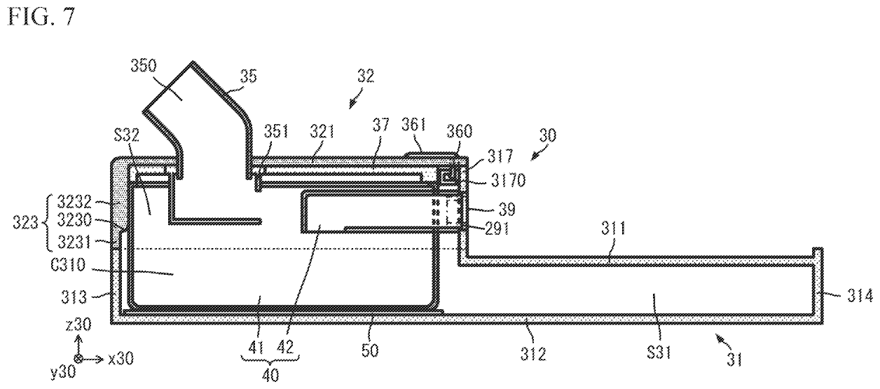

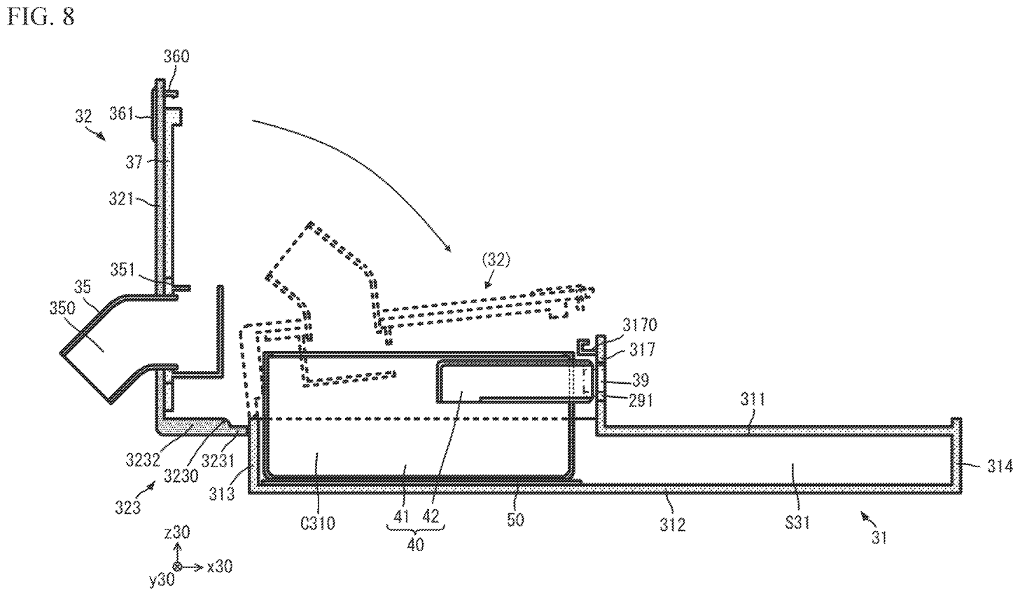

FIG. 4 is an external perspective view of the base unit according to the embodiment of the present disclosure. FIG. 5 is a top view of the base unit according to the embodiment of the present disclosure. FIG. 6 is a side view of the base unit according to the embodiment of the present disclosure. FIGS. 7 and 8 are each a sectional side view schematically illustrating the configuration of the base unit according to the embodiment of the present disclosure. FIGS. 6, 7, and 8 are figures viewed from the side of the long side wall of the base unit. FIGS. 6 and 7 illustrate a state in which the cover covers the upper surface side of the box body, and FIG. 8 illustrates a state in which the cover is rotating.

### Schematic Shape of Base Unit 30

As FIGS. 4, 5, 6, 7, and 8 illustrate, the base unit 30 includes a box body 31 and a cover 32. Schematically speaking, part of the upper surface side of the box body 31 is open. The cover 32 is rotatably connected to the box body 31. In the base unit 30, it is possible to switch between the state in which the cover 32 covers the opening of the box body 31 and the state in which the cover 32 does not cover the opening of the box body 31. The state in which the cover 32 covers the opening of the box body 31 is used, for example, during the usage as the CPAP apparatus 10, and the state in which the cover does not cover the opening of the box body 31 is used, for example, when the humidification chamber 40 accommodated in the base unit 30 is replenished with liquid.

### Box Body 31

The box body 31 includes an upper wall 311, a lower wall 312, a side wall 313, a side wall 314, a side wall 315, a side wall 316, and a side wall 317. The side wall 313 corresponds to a “first side wall” of the present disclosure, and the side wall 317 corresponds to a “second side wall” of the present disclosure.

The upper wall 311 and the lower wall 312 have different dimensions in the longitudinal direction (the x30-axis direction) and have substantially the same dimension in the lateral direction (the y30-axis direction). Part of the lower wall 312 in the longitudinal direction faces the upper wall 311.

The side wall 313 and the side wall 314 have substantially the same outside shape and face one another. The side wall 313 and the side wall 314 are parallel to the lateral direction of the base unit 30.

The side wall 315 and the side wall 316 have substantially the same outside shape and face one another. The side wall 315 and the side wall 316 are parallel to the longitudinal direction of the base unit 30.

The side wall 313, the side wall 314, the side wall 315, and the side wall 316 are connected to four sides of the lower wall 312. The side wall 314, part of the side wall 315 in the longitudinal direction, and part of the side wall 316 in the longitudinal direction are connected to three sides of the upper wall 311. Here, the upper wall 311 and the side wall 313 are apart from one another in the longitudinal direction of the box body 31.

The side wall 317 is disposed parallel to the side wall 313 and the side wall 314. The side wall 317 is connected to an end portion of the upper wall 311 on the opposite side from an end portion connected to the side wall 314. The side wall 317 has a shape extending further above (in the z30-axis direction) than the upper wall 311 with reference to the lower wall 312. That is, the side wall 317 has a shape protruding outward from a cuboid shape formed by the upper wall 311, the lower wall 312, the side wall 314, the side wall 315, the side wall 316, and the side wall 317. Thus, the box body 31 has a space S31A defined by the upper wall 311, the lower wall 312, the side wall 313, the side wall 314, the side wall 315, the side wall 316, and the side wall 317. At the same time, the box body 31 has a space S31B defined by the upper wall 311, the lower wall 312, the side wall 313, the side wall 315, the side wall 316, and the side wall 317. The space S31B is provided with an opening, on the upper side on which the upper wall 311 is disposed, between the upper wall 311 and the side wall 313 in plan view.

The side wall 314 has an inlet port 36. The inlet port 36 passes through the side wall 314 in the thickness direction.

The side wall 317 has an exhaust port 38 and an inlet port 39. The inlet port 39 corresponds to a “first opening” of the present disclosure. The exhaust port 38 passes through the inside of the side wall 317 and is communicated with the space S31A. The inlet port 39 passes through the side wall 317 in the thickness direction. The exhaust port 38 and the inlet port 39 are arranged adjacent to one another in the width direction of the side wall 317 (the y30-axis direction). The arrangement interval between the exhaust port 38 and the inlet port 39 is the same as the arrangement interval between the inlet port 281 and the exhaust port 291 of the main unit 20.

The side wall 317 includes a receiving member 3170 for a latch mechanism. The receiving member 3170 is formed on a wall surface of the side wall 317 on the opposite side from the upper wall 311 side.

### Cover 32

The cover 32 includes an upper wall 321, a side wall 323, a side wall 325, and a side wall 326. The side wall 323 corresponds to a “cover-side side wall” of the present disclosure. The outside shape of the upper wall 321 is substantially the same as the shape of the opening of the box body 31. The outside shape of the side wall 323 is substantially the same as the outside shape of the side wall 317 of the box body 31. The side wall 325 and the side wall 326 have substantially the same outside shape and face one another. The side wall 325 and the side wall 326 are parallel to the longitudinal direction of the base unit 30.

The side wall 323, the side wall 325, and the side wall 326 are connected to three sides of the upper wall 321. Thus, the cover 32 has a space S32 defined by the upper wall 321, the side wall 323, the side wall 325, and the side wall 326. Here, there is no wall facing the upper wall 311. Thus, the cover 32 has an opening on the side of the upper wall 311 on which the side wall 323, the side wall 325, and the side wall 326 are connected. In addition, there is no wall facing the side wall 323. Thus, the cover 32 has an opening on the side of the side wall 323 on which the upper wall 321, the side wall 325, and the side wall 326 are connected.

An air blowing pipe 35 is formed on the upper wall 321. The air blowing pipe 35 has a shape protruding outward from the upper wall 311. With the main unit 20 being mounted on the base unit 30, an opening 350 of the air blowing pipe 35 serves as, a final air blowing port through which desired air (blow) is supplied to a user. The opening 350 corresponds to a “second opening” of the present disclosure. In addition, an operator 361 for the latch mechanism is disposed on the upper wall 321. A hook 360 is connected to the operator 361. The hook 360 protrudes to the space S32 side relative to the upper wall 321.

An end portion of the side wall 323 on the opposite side from the end connected to the upper wall 321 is connected to the side wall 313 by using a hinge 390 including a rotation mechanism. The cover 32 is thus rotatably connected to the box body 31.

As FIG. 1 illustrates, the main unit 20 is mounted on the upper wall 311 of the box body 31 of the base unit 30. At this time, the side wall 203 of the main unit 20 is positioned close to or abuts on the side wall 317 of the base unit 30.

In this state, the exhaust port 38 of the base unit 30 and the inlet port 281 of the main unit 20 are communicated, and the cylindrical body 290 of the main unit 20 is inserted in the inlet port 39 of the base unit 30.

Note that, at the side wall 203 of the main unit 20, the side wall 317 of the base unit 30, and a proximity part (abutment part) therebetween, an elastic body, for example, is preferably disposed in a peripheral area of a part at which the exhaust port 38 of the base unit 30 and the inlet port 281 of the main unit 20 are communicated. Accordingly, the sealing performance relative to a gas flow passage between the main unit 20 and the base unit 30 is improved.

### Rotation Mechanism of Cover 32

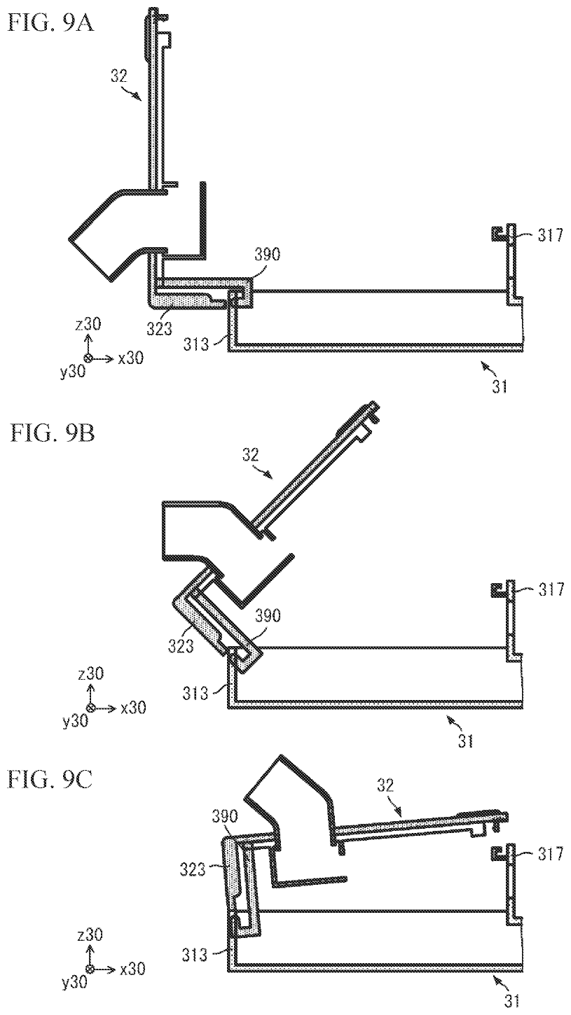

FIGS. 9A, 9B, and 9C are each a partial sectional side view of an example of the movement of the cover relative to the box body. There is illustrated a state in which the cover is closed in the order of FIG. 9A, FIG. 9B, and FIG. 9C.

As FIG. 9A, FIG. 9B, and FIG. 9C illustrate, in the rotation direction in which the upper wall 321 approaches the opening of the box body 31, the cover 32 is connected to the box body 31 such that the side wall 325 and the side wall 326 are positioned closer to the opening of the box body 31 than the upper wall 321. One end of the hinge 390 is rotatably connected to the side wall 313. The other end of the hinge 390 is fixed in proximity to the side wall 323 of the cover 32.

By using such a hinge 390, the cover 32 is rotated with a proximity position of the side wall 323 and the side wall 313 as a reference point as FIGS. 9A, 9B, and 9C illustrate. Thus, in the base unit 30, it is possible to select between the state in which the cover 32 covers the opening of the box body 31 illustrated in, for example, FIGS. 6 and 7 and the state in which the cover 32 does not cover the opening of the box body 31 illustrated in, for example, FIG. 8.

In the state in which the cover 32 covers the opening of the box body 31, the side wall 323 of the cover 32 is positioned close to an upper side (one side surrounding the opening) of the side wall 313 of the box body 31. The side wall 325 of the cover 32 is positioned close to an upper side (one side surrounding the opening) of the side wall 315 of the box body 31. The side wall 326 of the cover 32 is positioned close to an upper side (one side surrounding the opening) of the side wall 316 of the box body 31. The upper wall 321, the side wall 325, and the side wall 326 of the cover 32 are positioned close to three sides of the side wall 317 of the box body 31. Note that parts of the above-described walls close to the three sides of the side wall 317 may be in direct contact with the three sides of the side wall 317 or may be in contact with the three sides of the side wall 317 with an elastic body interposed therebetween.

Moreover, in the state in which the cover 32 covers the opening of the box body 31, the hook 360 of the cover 32 engages the receiving member 3170 of the box body 31. Thus, the cover 32 is fixed to the box body 31.

Due to this state, the space S32 of the cover 32 and the space S31B of the box body 31 are communicated through the opening of the space S31B. In this state, an accommodation space constituted by the space S31B and the space S32 is formed in the base unit 30. Here, with the latch mechanism constituted by the hook 360 and the receiving member 3170, the base unit 30 enables such a state of the accommodation space to be maintained more reliably.

The inside space of the base unit 30 formed as described above is communicated with the outside of the base unit 30 through the air blowing pipe 35, the inlet port 36, the exhaust port 38, and the inlet port 39.

### Humidification Chamber 40

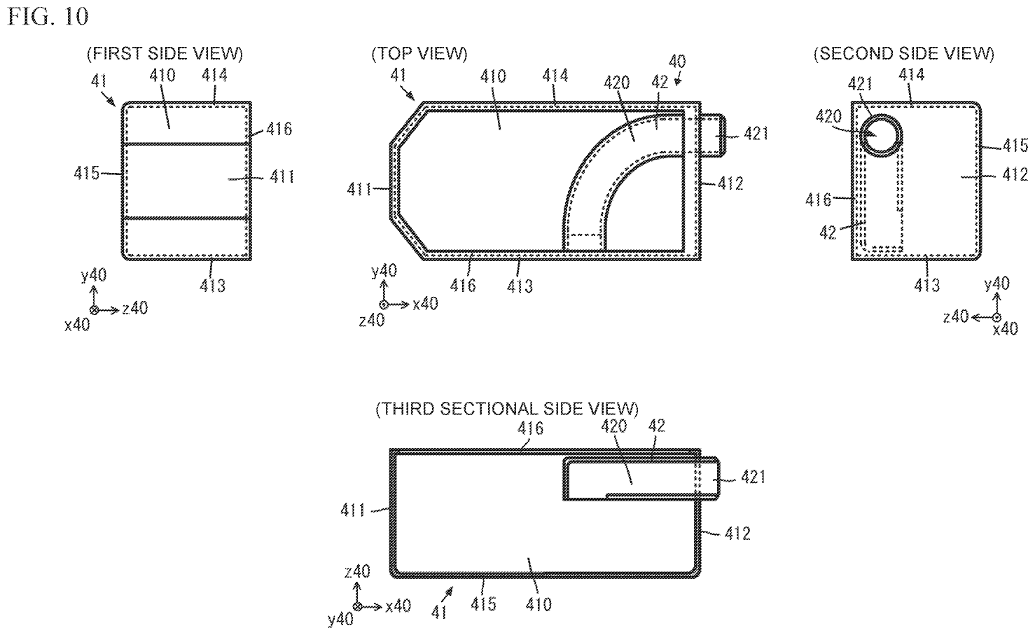

FIG. 10 is a four orthogonal view of the humidification chamber 40 according to the embodiment of the present disclosure. FIG. 10 includes a top view, a first side view, a second side view, and a third side view of the humidification chamber.

As FIG. 10 illustrates, the humidification chamber 40 includes a chamber main body 41 and a tubular body 42.

The chamber main body 41 includes a chamber side wall 411, a chamber side wall 412, a chamber side wall 413, a chamber side wall 414, a chamber lower wall 415, and an upper side frame 416. The chamber side wall 411 corresponds to a “third side wall” of the present disclosure, and the chamber side wall 412 corresponds to a “fourth side wall” of the present disclosure.

Part of the chamber side wall 411 and part of the chamber side wall 412 face one another. The chamber side wall 411 and the chamber side wall 412 are parallel to the lateral direction of the humidification chamber 40 (the y40-axis direction).

The chamber side wall 413 and the chamber side wall 414 have substantially the same outside shape and face one another. The chamber side wall 413 and the chamber side wall 414 are parallel to the longitudinal direction of the humidification chamber 40 (the x40-axis direction).

The chamber lower wall 415 is parallel to the longitudinal direction (the x40-axis direction) and the lateral direction (the y40-axis direction) of the humidification chamber 40.

The chamber lower wall 415 is connected to the chamber side wall 411, the chamber side wall 412, the chamber side wall 413, and the chamber side wall 414. Thus, the chamber main body 41 has a space 410 defined by the chamber side wall 411, the chamber side wall 412, the chamber side wall 413, the chamber side wall 414, and the chamber lower wall 415.

The chamber main body 41 includes the upper side frame 416 at an upper end (on the opposite side from the chamber lower wall 415). The upper side frame 416 is connected to the chamber side wall 411, the chamber side wall 412, the chamber side wall 413, and the chamber side wall 414 and has an opening in the center. Through the opening, the space 410 of the chamber main body 41 is communicated with the outside of the chamber main body 41.

The tubular body 42 includes a bent part or a curved part at some midpoint in a direction in which the inside space thereof extends. One end of the tubular body 42 in the extension direction of the tubular body 42 protrudes outside the chamber main body 41. The other end of the tubular body 42 in the extension direction of the tubular body 42 is disposed inside the space 410 of the chamber main body 41. Through the tubular body 42, the space 410 of the chamber main body 41 is communicated with the outside of the chamber main body 41 on the chamber side wall 412 side.

The other end of the tubular body 42 in the extension direction of the tubular body 42 is preferably disposed in proximity to the chamber side wall 413.

The tubular body 42 is made of, for example, resin having elasticity. At least a distal end portion 421 that is the one end of the tubular body 42 in the extension direction of the tubular body 42 and protrudes from the chamber main body 41, is made of deformable resin.

### Mounting Mechanism of Humidification Chamber 40 on Base Unit 30

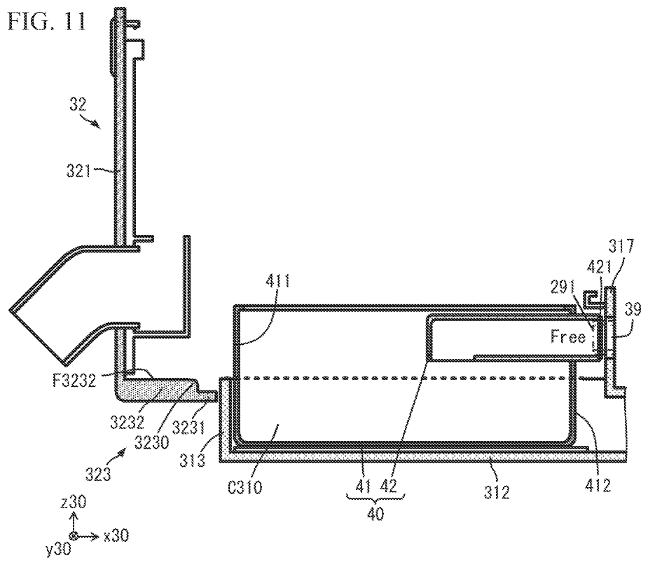

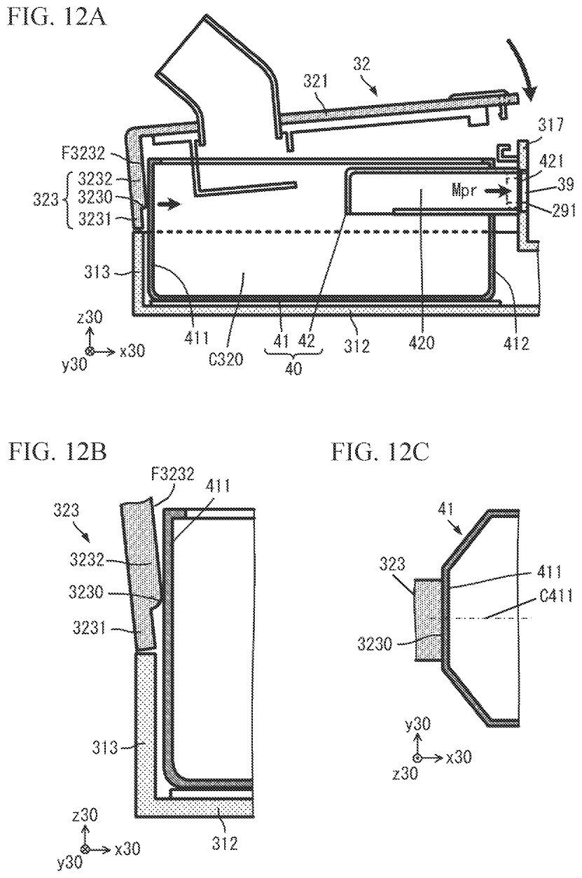

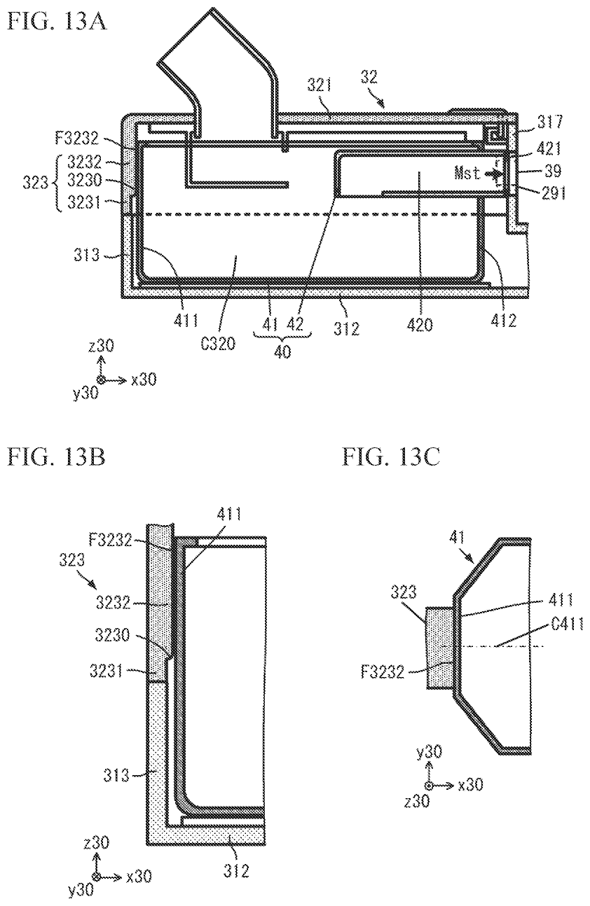

FIG. 11 is a partial sectional side view of a first state of the mounting state of the humidification chamber on the base unit. FIG. 12A is a partial sectional side view of a second state of the mounting state of the humidification chamber on the base unit, FIG. 12B is a sectional side view of an enlarged connection area of a side wall of the cover and the humidification chamber in the second state, and FIG. 12C is a top view of the enlarged connection area of the side wall of the cover and the humidification chamber in the second state. FIG. 13A is a partial sectional side view of a third state of the mounting state of the humidification chamber on the base unit, FIG. 13B is a sectional side view of the enlarged connection area of the side wall of the cover and the humidification chamber in the third state, and FIG. 13C is a top view of the enlarged connection area of the side wall of the cover and the humidification chamber in the third state.

### First State

In the first state illustrated in FIG. 11, the cover 32 leaves the upper side of the opening of the box body 31 open. In this state, the humidification chamber 40 can be accommodated into the space S31B in the box body 31 from outside. In addition, the humidification chamber 40 can easily be replenished with liquid with the humidification chamber 40 being disposed in the box body 31.

At this time, the humidification chamber 40 is disposed in the box body 31 such that the chamber side wall 411 is positioned on the side wall 313 side and the side wall 323 side, and the chamber side wall 412 is positioned on the side wall 317 side. At this time, the distal end portion 421 of the tubular body 42 of the humidification chamber 40 is positioned close to the inlet port 39 of the side wall 317.

The humidification chamber 40 is not fixed to the base unit 30 in this state. Thus, a user can easily remove the humidification chamber 40 from the base unit 30, and, to put it the other way around, the humidification chamber 40 can easily be placed into the space S31B of the base unit 30. In addition, in this state, unnecessary external force is not applied to the distal end portion 421 of the tubular body 42, and unnecessary abrasion and degradation of the distal end portion 421 of the tubular body 42 can thereby be suppressed.

### Second State

In the second state illustrated in FIGS. 12A, 12B, and 12C, the cover 32 in the first state is rotated, and the upper wall 321 is approaching the opening of the box body 31.

Here, specifically, the side wall 323 has the following shape. The side wall 323 includes a thin portion 3231 and a thick portion 3232. The thin portion 3231 and the thick portion 3232 are connected in the order of the thick portion 3232 and the thin portion 3231 from the upper wall 321 side. Conversely speaking, the thin portion 3231 and the thick portion 3232 are connected in the order of the thin portion 3231 and the thick portion 3232 from the side of the end portion connected to the side wall 313 of the box body 31. That is, the thin portion 3231 and the thick portion 3232 have a connection part at a mid-position in the extension direction of the side wall 323 (a direction orthogonal to the width direction and the thickness direction). In other words, such a connection part is a part in which there is a thickness change from the thin portion 3231 to the thick portion 3232.

The outer surface of the thin portion 3231 and the outer surface of the thick portion 3232 are flush with one another. Thus, the thick portion 3232 protrudes closer to the space S32 side (the inner side) of the cover 32 than the thin portion 3231, with the connection part interposed therebetween. With such a configuration, the side wall 323 includes a ridge portion 3230 protruding to the inner side of the cover 32, at a mid-position in the extension direction thereof. The ridge portion 3230 has a predetermined length in the width direction of the side wall 323 (the y30-axis direction). The part constituted by the thick portion 3232 and the ridge portion 3230 corresponds to a “pressing portion (protruding portion)” of the present disclosure.

With the side wall 323 including the thin portion 3231, the thick portion 3232, and the ridge portion 3230 protruding to the inner side, the ridge portion 3230 comes into contact with the outer surface of the chamber side wall 411 of the humidification chamber 40 when the cover 32 approaches the opening of the box body 31 and is positioned at a predetermined distance therefrom.

When the cover 32 in this state continues rotating, the ridge portion 3230 pushes the chamber side wall 411 as FIG. 12A illustrates. In this state, the humidification chamber 40 is not fixed to the box body 31. Thus, the humidification chamber 40 is moved to the side wall 317 side by receiving the force pushing the humidification chamber 40 from the side wall 323. The distal end portion 421 of the tubular body 42 is then pressed against the face of the exhaust port 291 of the cylindrical body 290. A pressing amount Mpr of such pressing is equivalent to a magnitude with which the distal end portion 421 is deformed, and the distal end portion 421 is flattened at such a predetermined amount. Thus, an inside space 420 of the tubular body 42 and the inlet port 39 are communicated while being sealed off from the outside.

At this time, by the distal end portion 421 being flattened at the pressing amount Mpr, the force bringing the tubular body 42 and the face of the exhaust port 291 of the cylindrical body 290 into close contact is increased, and the sealing performance of the inside space 420 of the tubular body 42 and the exhaust port 291 and a communication part thereof is further increased.

In addition, as FIG. 12C illustrates, in the second state, the ridge portion 3230 is in linear contact with the chamber side wall 411 such that a position of the center in the width direction of the humidification chamber 40 (a central axis C411 in the width direction of the chamber side wall 411) is included. Thus, uneven contact of the side wall 323 with the chamber side wall 411 can be suppressed, and the state in which the distal end portion 421 of the tubular body 42 is in contact with the cylindrical body 290 while surrounding the exhaust port 291 can be achieved more reliably.

In addition, as FIGS. 12A and 12B illustrate, in the second state, the ridge portion 3230 is preferably in contact with a position in the upper half of the chamber side wall 411. Accordingly, it is possible to more reliably suppress rising-up of the humidification chamber 40 caused by the contact of the ridge portion 3230 with the chamber side wall 411.

### Third State

In the third state illustrated in FIGS. 13A, 13B, and 13C, the cover 32 covers the opening of the box body 31.

In this state, as FIGS. 13A, 13B, and 13C illustrate, an inner wall surface F3232 of the thick portion 3232 of the side wall 323 is in contact with the chamber side wall 411. At this time, a pressing amount Mst acts on the distal end portion 421 of the tubular body 42. Thus, the sealed communication state of the inside space 420 of the tubular body 42 and the exhaust port 291 is maintained.

Thus, the humidification chamber 40 is fixed inside the base unit 30 while the sealed communication state of the inside space 420 of the tubular body 42 and the exhaust port 291 is maintained.

Moreover, the hook 360 of the cover 32 and the receiving member 3170 of the box body 31 are engaged to achieve a fixed state with the latch mechanism, and the cover 32 is thereby fixed to the box body 31. Thus, the position of the side wall 323 relative to the box body 31 is fixed, and the state in which the inner wall surface F3232 of the thick portion 3232 is in contact with the chamber side wall 411 is maintained. Accordingly, the humidification chamber 40 is fixed inside the base unit 30 while the sealed communication state of the inside space 420 of the tubular body 42 and the exhaust port 291 is further stably maintained.

At this time, the pressing amount Mst is made smaller than the pressing amount Mpr by appropriately setting the shape of the ridge portion 3230, the shape of the thick portion 3232, and the distances between the portions and the center of the rotation. Thus, an amount by which the distal end portion 421 is flattened in the third state can be reduced. Accordingly, the distal end portion 421 of the tubular body 42 can be suppressed from being degraded.

In addition, as FIG. 13C illustrates, in the third state, the inner wall surface F3232 is in linear contact with the chamber side wall 411 such that the position of the center in the width direction of the humidification chamber 40 (the central axis C411 in the width direction of the chamber side wall 411) is included. Thus, uneven contact of the side wall 323 with the chamber side wall 411 can be suppressed, and the fixed state of the humidification chamber 40 to the base unit 30 can further be stabilized.

In addition, as FIGS. 13A and 13B illustrate, in the third state, the inner wall surface F3232 is preferably in contact with a position in the upper half of the chamber side wall 411. Thus, rising-up of the humidification chamber 40 can be suppressed more reliably, and the humidification chamber 40 can be fixed to the base unit 30 in an appropriate position.

When the main unit 20 starts operating in this state, the fan of the main unit 20 operates to start air blowing. Thus, gas is supplied inside the base unit 30 from the inlet port 36 of the base unit 30 and is supplied to the fan inside the main unit 20 through the exhaust port 38 and the inlet port 281. The gas that has been delivered from the fan is introduced into the tubular body 42 of the humidification chamber 40 through the cylindrical body 290 and the inlet port 39. The gas that has been introduced into the tubular body 42 is introduced into the space 410. The liquid stored in the chamber main body 41 is heated by a heater 50. Thus, an upper part of the chamber main body 41 is supplied with the gas having a predetermined pressure and humidity. The gas having a predetermined pressure and humidity is discharged outside from the air blowing pipe 35 and is supplied to a user.

In such an operation, with such an above-described fixed structure of the humidification chamber 40, gas leaking when gas is supplied from the main unit 20 to the humidification chamber 40 can be suppressed. Thus, the CPAP apparatus 10 can stably supply the gas having a predetermined pressure and humidity to a user.

In addition, in the CPAP apparatus 10, the fixed structure and the release structure of the humidification chamber 40 are achieved with the simple configuration in which pressing relative to the humidification chamber 40 is changed by using the side wall of the cover 32. Thus, no intricate configuration as in the related art is required. Accordingly, downsizing of the CPAP apparatus 10 is achieved with such a simple configuration while the humidification chamber 40 is disposed at appropriate positions when in use and when water replenishment is performed.

In addition, as FIGS. 5, 6, 7, and other figures illustrate, the base unit 30 includes a seal member 37 at the back side (on the space S32 side) of the upper wall 321 of the cover 32. The seal member 37 is constituted by a sheet-shaped elastic body and has a shape with which the opening of the air blowing pipe 35 on the space S32 side is not closed. Moreover, the seal member 37 is disposed at a position at which the seal member 37 abuts on the upper side frame 416 of the chamber main body 41 with the cover 32 covering the opening of the box body 31.

In the above-described third state as illustrated in FIG. 13, the seal member 37 abuts on and is in close contact with the upper side frame 416 of the chamber main body 41. Thus, the sealing performance of the humidification chamber 40 is improved, and the CPAP apparatus 10 can further stably supply the gas having a predetermined pressure and humidity to a user.

In addition, as FIGS. 5, 6, 7, and other figures illustrate, a rectification member 351 is disposed at a cavity, to the space S32, of the air blowing pipe 35 of the cover 32. When viewed from the lower wall 312 side, the rectification member 351 covers the opening of the air blowing pipe 35. The CPAP apparatus 10 can stabilize the airflow discharged from the air blowing pipe 35 by using such a rectification member 351.

Note that, in the above description, no specific shape of the ridge portion 3230 is given. The ridge portion 3230 may have an outside shape whose cross-section has a corner; however, the outside shape thereof preferably has an arc-shaped cross-section. Accordingly, the side wall 323 can be suppressed from excessively pressing the humidification chamber 40. Thus, in the CPAP apparatus 10, the distal end portion 421 of the tubular body 42 can be suppressed from being undesirably degraded.

In addition, the above description illustrates the form in which the inner wall surface F3232 is a plane (the outside shape of a cross-section of the inner wall surface F3232 is a linear shape). However, as long as the humidification chamber 40 can be held at a predetermined position, the outside shape of a cross-section of the inner wall surface F3232 may have a desired curved line or an uneven part.

In addition, when viewed in a direction orthogonal to the side wall 323 (when viewed from the pressing portion to the inlet port 39 side), a part of the side wall 323 corresponding to the pressing portion (a part constituted by the thick portion 3232 and the ridge portion 3230) preferably overlaps the inlet port 39 and the cylindrical body 290 (the exhaust port 291) with the main unit 20 being mounted. Accordingly, in the CPAP apparatus 10, no excessive force exerted in the rotation direction is applied to the humidification chamber 40, and the humidification chamber 40 can stably be pressed in the horizontal direction.

(1) A CPAP apparatus includes: a main body including a box body whose upper surface side is open, a cover that covers the upper surface side of the box body, and a first opening and a second opening; and a humidification chamber accommodatable inside the main body and disposed between the first opening and the second opening, in which the box body includes a lower wall, and a first side wall and a second side wall that are arranged with a space therebetween in which the humidification chamber is capable of being disposed, the cover includes a main wall having a plate shape and a cover-side side wall connected to the main wall and rotatably connected to the first side wall, the humidification chamber includes: a main body that stores the liquid and has a box shape that is open upward, the main body including a third side wall and a fourth side wall; and a tubular body passing through the fourth side wall, the humidification chamber is accommodated in the box body with the first side wall and the third side wall facing one another and with the second side wall and the fourth side wall facing one another, the second side wall of the box body has a through hole through which the tubular body and an inside of the main body are communicable with one another with the humidification chamber being accommodated in the box body, and the cover-side side wall includes a pressing portion that is in contact with the third side wall when rotated.

(2) The CPAP apparatus according to (1), in which the cover-side side wall includes a thick portion and a thin portion, the thin portion and the thick portion are positioned in this order from a side connected to the first side wall, and the thick portion constitutes the protruding portion.

(3) The CPAP apparatus according to (2), in which a thickness change part in which there is a thickness change from the thin portion to the thick portion is in contact with the third side wall during rotation, and a surface of the thick portion is in contact with the third side wall with the cover being fixed while covering the box body.

(4) The CPAP apparatus according to (3), in which the thickness change part has a cross-section having an arc-shaped outside shape.

(5) The CPAP apparatus according to (3) or (4), in which the thickness change part has a predetermined length in a width direction of the cover-side side wall.

(6) The CPAP apparatus according to (5), in which the thickness change part is in linear contact with the third side wall such that a center in a width direction of the third side wall is included.

(7) The CPAP apparatus according to any one of (1) to (6), in which an end portion of the main wall of the cover on an opposite side from an end portion to which the cover-side side wall is connected has a latch mechanism with which fixing and releasing relative to the box body are selectable.

(8) The CPAP apparatus according to any one of (1) to (7), in which the main wall of the cover is provided with a seal member that closes an opening on an upper side of the humidification chamber with the cover being fixed while covering the box body.

(9) The CPAP apparatus according to any one of (1) to (8), in which the tubular body includes an elastic body.

(10) The CPAP apparatus according to any one of (1) to (9), in which the pressing portion overlaps the first opening in a side view from the pressing portion to the first opening.

(11) The CPAP apparatus according to any one of (1) to (10), in which the tubular body is mounted on the main body and partially protrudes outside from the fourth side wall.

(12) The CPAP apparatus according to any one of (1) to (11), in which the pressing portion is a protruding portion formed on an inner surface of the cover-side side wall.

What is claimed is:

1. A CPAP apparatus comprising:
a main body including a box body having an upper surface side opened, a cover covering the upper surface side of the box body, and a first opening and a second opening; and
a humidification chamber accommodatable inside the main body and disposed between the first opening and the second opening, wherein
the box body includes
a lower wall, and a first side wall and a second side wall, the first side wall and the second side wall being arranged with a space therebetween, and the space being sufficient to arrange the humidification chamber therein,
the cover includes
a main wall having a plate shape and a cover-side side wall connected to the main wall and rotatably connected to the first side wall,
the humidification chamber includes:
a main body configured to store liquid and having a box shape opened upward, the main body including a third side wall and a fourth side wall; and
a tubular body passing through the fourth side wall,
the humidification chamber is accommodated in the box body with the first side wall and the third side wall facing one another and with the second side wall and the fourth side wall facing one another,
the second side wall of the box body has a through hole through which the tubular body and an inside of the main body are communicable with one another with the humidification chamber being accommodated in the box body, and
the cover-side side wall includes a pressing portion being in contact with the third side wall when rotated.

2. The CPAP apparatus according to claim 1, wherein
the cover-side side wall includes a thick portion and a thin portion,
the thin portion and the thick portion are positioned in this order from a side connected to the first side wall, and
the thick portion constitutes a protruding portion.

3. The CPAP apparatus according to claim 2, wherein
a thickness change part having a thickness changed from the thin portion to the thick portion is in contact with the third side wall during the rotation, and
a surface of the thick portion is in contact with the third side wall with the cover being fixed while covering the box body.

4. The CPAP apparatus according to claim 3, wherein
the thickness change part has a cross-section having an arc-shaped outside shape.

5. The CPAP apparatus according to claim 3, wherein
the thickness change part has a predetermined length in a width direction of the cover-side side wall.

6. The CPAP apparatus according to claim 5, wherein
the thickness change part is in linear contact with the third side wall such that a center in a width direction of the third side wall is included.

7. The CPAP apparatus according to claim 1, wherein
an end portion of the main wall of the cover on an opposite side from an end portion to which the cover-side side wall is connected has a latch mechanism with which fixing and releasing relative to the box body are selectable.

8. The CPAP apparatus according to claim 1, wherein
the main wall of the cover is provided with a seal member closing an opening on an upper side of the humidification chamber with the cover being fixed while covering the box body.

9. The CPAP apparatus according to claim 1, wherein
the tubular body includes an elastic body.

10. The CPAP apparatus according to claim 1, wherein
the pressing portion overlaps the first opening in a side view from the pressing portion to the first opening.

11. The CPAP apparatus according to claim 1, wherein
the tubular body is mounted on the main body and partially protrudes outside from the fourth side wall.

12. The CPAP apparatus according to claim 1, wherein
the pressing portion is a protruding portion provided on an inner surface of the cover-side side wall.
